(19) United States
(12) Patent Application Publication
(10) Pub. No.: US 2004/0199054 A1
(43) Pub. Date: Oct. 7, 2004

(54) Magnetically propelled capsule endoscopy

(76) Inventor: Glenn Mark Wakefield, Tempe, AZ (US)

Correspondence Address: Glenn Wakefield, 1416 East Carmen Street, Tempe, AZ, 85283, US

(21) Appl. No.: 10/406,336

(22) Filed: Apr. 3, 2003

(51) Int. Cl. A61B 1/00

(52) U.S. Cl. 600/160

(57) ABSTRACT

Magnetically propelled capsule endoscopy provides for the medical examination of the gastrointestinal tract. After the capsule is swallowed, it will be steered throughout the gastrointestinal tract by an externally generated magnetic field. This device will provide real time imagery and position along with the ability to perform tissue biopsy/excision. This device can be applied to the gastrointestinal tract, reproductive tract, trachea/lungs, vascular system or any accessible body cavity.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### BACKGROUND OF THE INVENTION

This patent application applies to the field of medical imaging and tissue biopsy/excision.

Traditionally the digestive tract has been examined by the upper gastrointestinal endoscope and the lower gastrointestinal endoscope and most recently by the capsule endoscope. Upper and lower gastrointestinal endoscopy usually require an anesthetic and are limited by the inability to examine the small intestine. Capsule endoscopy does not practically allow for real time imaging and precludes tissue biopsy/excision. The upper and lower gastrointestinal endoscopy and capsule endoscopy provide a limited range of viewing/access angles.

### BRIEF SUMMARY OF THE INVENTION

Magnetically propelled capsule endoscopy provides for the medical examination of the gastrointestinal tract. After the capsule is swallowed, it will be steered throughout the gastrointestinal tract by an externally generated magnetic field. This device will provide real time imagery and position data along with the ability to perform tissue biopsy/excision. This device can be applied to the gastrointestinal tract, reproductive tract, trachea/lungs, vascular system or any accessible body cavity.

### BRIEF DESCRIPTION OF THE SEVERAL VIEWS OF THE DRAWING

Not Applicable

### DETAILED DESCRIPTION OF THE INVENTION

Magnetically propelled capsule endoscopy allows for a fast, comprehensive medical examination of the digestive tract, reproductive tract, trachea/lungs, vascular system or any accessible body cavity. The capsule is swallowed or appropriately placed in the body. Contained within the small capsule will be:

(#1 possibility)—magnets for movement and directional control; LEDs for lighting and camera for visualization; rf source and receiver to transmit pictures and to communicate; gyroscope/accelerometer for positional information; mechanical equipment for tissue biopsy/excision or other procedures; power source; electronics;

(#2 possibility)—magnets for movement and directional control; fiber optic light source, fiber optic imaging lens, power lines, communication lines, air hose, and water hose extending out the back of the capsule and connecting to the external control instrument; gyroscope/accelerometer for positional information; mechanical equipment for tissue biopsy/excision or other procedures; electronics.

All of the parts in #1 possibility and #2 possibility, other than the magnets, will be composed of nonmagnetic material or magnetically shielded as required. The #1 possibility is a capsule with no physical connection to the outside in contrast to the #2 possibility that is a capsule that has a physical connection to the outside. The choice of physical connection or not, capsule size and capabilities will vary depending on the part of the body that is being examined by the medical practitioner.

The magnets contained within the capsule will be bathed in an externally generated magnetic field. The external field is created by inscribing six metal rings on the six faces of an imaginary cube. Current is running through each of the six rings which effectively allows a pair of rings to control each dimension. The metal rings are composed of many windings of appropriately coated wire. The patient undergoing the medical examination will be placed inside the six ringed structure. The capsule will be moved by appropriately changing the current within each ring. As necessary, the actual rings will be moved in conjunction with the changing current. The medical practitioner, using visual feedback, will guide the capsule throughout the body part under examination. The magnetic field strength will be adjusted appropriately for direction change, movement or extra stationary stability for tissue biopsy/excision or other procedures.

The gyroscope/accelerometer will provide the ability to map in real time the positional progress of the capsule. A positional map will be created of the entire exam, areas of interest can be marked, and visual images will be generated corresponding to each positional location of the capsule. A computer generated three dimensional fly through can be created based on the data collected from the examination. All of this data can be appropriately stored for future reference and for comparison with other medical exams.

I claim:

1. Magnetically propelled capsule endoscopy allows for a fast, comprehensive medical examination of the digestive tract, reproductive tract, trachea/lungs, vascular system or any accessible body cavity. The capsule is swallowed or appropriately placed in the body. Contained within the small capsule will be:
(#1 possibility)—magnets for movement and directional control; LEDs for lighting and camera for visualization; rf source and receiver to transmit pictures and to communicate; gyroscope/accelerometer for positional information; mechanical equipment for tissue biopsy/excision or other procedures; power source; electronics;
(#2 possibility)—magnets for movement and directional control; fiber optic light source, fiber optic imaging lens, power lines, communication lines, air hose, and water hose extending out the back of the capsule and connecting to the external control instrument; gyroscope/accelerometer for positional information; mechanical equipment for tissue biopsy/excision or other procedures; electronics.
All of the parts in #1 possibility and #2 possibility, other than the magnets, will be composed of nonmagnetic material or magnetically shielded as required. The #1 possibility is a capsule with no physical connection to the outside in contrast to the #2 possibility that is a capsule that has a physical connection to the outside. The choice of physical connection or not, capsule size and capabilities will vary depending on the part of the body that is being examined by the medical practitioner.
The magnets contained within the capsule will be bathed in an externally generated magnetic field. The external field is created by inscribing six metal rings on the six faces of an imaginary cube. Current is running through each of the six rings which effectively allows a pair of rings to control each dimension. The metal rings are composed of many windings of appropriately coated wire. The patient undergoing the medical examination will be placed inside the six ringed structure. The capsule will be moved by appropriately changing the current within each ring. As necessary, the actual rings will be moved in conjunction with the changing current. The medical practitioner, using visual feedback, will guide the capsule throughout the body part under examination. The magnetic field strength will be adjusted appropriately for direction change, movement or extra stationary stability for tissue biopsy/excision or other procedures.
The gyroscope/accelerometer will provide the ability to map in real time the positional progress of the capsule. A positional map will be created of the entire exam, areas of interest can be marked, and visual images will be generated corresponding to each positional location of the capsule. A computer generated three dimensional fly through can be created based on the data collected from the examination. All of this data can be appropriately stored for future reference and for comparison with other medical exams.
